(19) United States
(12) Patent Application Publication
(10) Pub. No.: US 2026/0240524 A1
(43) Pub. Date: Aug. 20, 2026

(54) ULTRASOUND DIAGNOSTIC APPARATUS

(71) Applicant: FUJIFILM Corporation, Tokyo (JP)

(72) Inventors: Jun YASUDA, Tokyo (JP); Misaki MARUYAMA, Tokyo (JP); Teiichiro IKEDA, Tokyo (JP); Nobuhiko FUJII, Tokyo (JP); Natsumi DANNO, Tokyo (JP)

(73) Assignee: FUJIFILM Corporation, Tokyo (JP)

(21) Appl. No.: 19/444,225

(22) Filed: Jan. 9, 2026

(30) Foreign Application Priority Data
Feb. 14, 2025 (JP) 2025-022392

(51) Int. Cl. A61B 8/00; G01S 7/52

(52) U.S. Cl. CPC A61B 8/481; A61B 8/461; A61B 8/5207; G01S 7/52057

(57) ABSTRACT

An ultrasound diagnostic apparatus 10 includes a biological parameter calculation processing unit 1042 that acquires a biological parameter set representing an ultrasound propagation state in a subject 300 by performing pilot transmission, a sound pressure distribution simulation execution unit 1102 that executes a sound pressure distribution simulation by using one transmission parameter set extracted from an ultrasound transmission condition storage unit 118 as a transmission condition and the biological parameter set as an input to estimate a sound pressure distribution, and a sound pressure distribution analysis unit 1104 that selects a transmission parameter set for obtaining an optimal contrast image by analyzing a plurality of sound pressure distributions estimated by a plurality of times of the sound pressure distribution simulation using different transmission parameter sets, and determines the selected transmission parameter set as a transmission condition used for contrast-enhanced ultrasonography.

### CROSS-REFERENCE TO RELATED APPLICATIONS

The present application claims priority under 35 U.S.C. §119 to Japanese Patent Application No. 2025-022392, filed on Feb. 14, 2025. The above application is hereby expressly incorporated by reference, in its entirety, into the present application.

### BACKGROUND OF THE INVENTION

### Field of the Invention

The present disclosure relates to an ultrasound diagnostic apparatus, particularly, an ultrasound diagnostic apparatus used for contrast-enhanced ultrasonography.

### Description of the Related Art

contrast-enhanced ultrasonography (CEUS) is a technology that allows imaging of blood vessels in a living body by causing a contrast agent injected intravenously into the living body to vibrate vigorously by an ultrasound beam and extracting a nonlinear component such as a harmonic wave from a received echo. The bubble-like contrast agent has three types of behaviors of reflection, resonance, and collapse due to irradiation with ultrasound waves, and the behaviors depend on a sound pressure of the transmitted ultrasound beam. In order to display the blood vessel in a high-contrast and favorable image, it is preferable that the sound pressure of the ultrasound waves falls between a contrast threshold value defined as a lower limit threshold value at which imaging can be performed with at least a predetermined level of tissue contrast and a breakage threshold value as an upper limit threshold value that can exist without breaking the contrast agent, in the entire depth direction in the subject. Since the sound pressure of the transmitted ultrasound beam depends on the transmission condition, it is necessary to adjust the transmission condition such that the sound pressure is within the above-described threshold values.

### SUMMARY OF THE INVENTION

However, even in a case in which the contrast-enhanced ultrasonography is performed under the same transmission condition, the vibration state of the contrast agent varies depending on a difference in body shape of the subject or the like. In addition, even in the same subject, the vibration state of the contrast agent may dynamically change depending on a location of a tissue in which the contrast agent is present and a condition of the subject such as blood pressure and body temperature during the examination. Therefore, in order to generate the ultrasound image representing a favorable contrast agent distribution over a wide range in the depth direction in the subject, it is necessary to appropriately set the transmission condition used in the contrast-enhanced ultrasonography each time.

An object of the present disclosure is to easily image a contrast agent appropriately over a wide range in a depth direction in a subject. Alternatively, an object of the present disclosure is to obtain a transmission condition suitable for contrast-enhanced ultrasonography of each subject.

An ultrasound diagnostic apparatus according to the present disclosure comprises a processor, in which the processor is configured to: select a provisional measurement condition from a plurality of measurement conditions that cause different transmission beams to be generated; generate an intensity distribution by performing a simulation or preliminary transmission and reception based on the provisional measurement condition, the intensity distribution being a sound pressure distribution along a depth direction in a specific subject or a distribution related to a harmonic wave in which the sound pressure distribution is reflected; calculate an evaluation value representing a size of a portion satisfying an appropriate intensity condition for contrast agent imaging in the intensity distribution; select a main measurement condition from the plurality of measurement conditions based on a plurality of evaluation values each corresponding to the plurality of measurement conditions; and generate an ultrasound image representing a contrast agent distribution in the specific subject based on reception information obtained from the specific subject by performing transmission and reception for contrast-enhanced ultrasonography based on the main measurement condition.

In addition, the appropriate intensity condition may include an intensity lower limit threshold value for ensuring a minimum contrast of the ultrasound image and an intensity upper limit threshold value for avoiding contrast agent breakage, and the portion satisfying the appropriate intensity condition may be a portion of the sound pressure distribution that falls between the intensity lower limit threshold value and the intensity upper limit threshold value.

In addition, the measurement condition may be a transmission parameter set that is a transmission condition for generating a transmission beam.

In addition, the processor may be configured to: perform preliminary transmission and reception for obtaining a biological parameter set representing an ultrasound propagation state in the specific subject based on the reception information obtained from the specific subject; and execute the simulation by using the transmission parameter set and the biological parameter set as input parameters to estimate the sound pressure distribution along the depth direction in the specific subject.

In addition, the appropriate intensity condition may have an intensity lower limit threshold value for ensuring a desired contrast of the ultrasound image, and the portion satisfying the appropriate intensity condition may be a portion of the distribution related to the harmonic wave that is equal to or higher than the intensity lower limit threshold value.

In addition, the measurement condition may be a combination of a transmission parameter set, which is a transmission condition in a case of performing the preliminary transmission and reception and the transmission and reception for the contrast-enhanced ultrasonography, and a reception parameter set that is a reception condition.

In addition, the processor may be configured to calculate the evaluation value based on a display range of the ultrasound image or a range of interest in the depth direction in the specific subject.

According to the present disclosure, the contrast agent can be easily imaged appropriately over a wide range in the depth direction in the subject. Alternatively, according to the present disclosure, a transmission condition suitable for the contrast-enhanced ultrasonography of each subject can be obtained.

### BRIEF DESCRIPTION OF THE DRAWINGS

FIG. 1 is a block diagram showing a schematic configuration of an ultrasound diagnostic apparatus according to Embodiment 1.

FIG. 2 is a flowchart showing a method of determining a transmission condition according to Embodiment 1.

The left diagram in FIG. 3 is a diagram showing a sound pressure distribution with respect to a depth direction of a subject in two dimensions, and the right diagram in FIG. 3 is a diagram showing a level of a sound pressure obtained from the sound pressure distribution shown in the left diagram .

The left diagram in FIG. 4 is a diagram showing a specific example of a site of the subject to be examined, and the right diagram in FIG. 4 is a diagram showing a level of a sound pressure obtained from a sound pressure distribution obtained by executing a sound pressure distribution simulation on the site shown in the left diagram .

FIG. 5 is a block diagram showing a schematic configuration of an ultrasound diagnostic apparatus according to Embodiment 2.

FIG. 6 is a flowchart showing a method of determining a transmission/reception condition according to Embodiment 2.

The left diagram in FIG. 7 is a diagram schematically showing a harmonic power distribution with respect to a depth direction of a subject, and the right diagram in FIG. 7 is a diagram showing a level of a harmonic power obtained from the harmonic power distribution shown in the left diagram.

### DESCRIPTION OF THE PREFERRED EMBODIMENTS

Hereinafter, embodiments according to the present disclosure will be described with reference to the drawings.

### EMBODIMENT 1

### Configuration of Ultrasound Diagnostic Apparatus

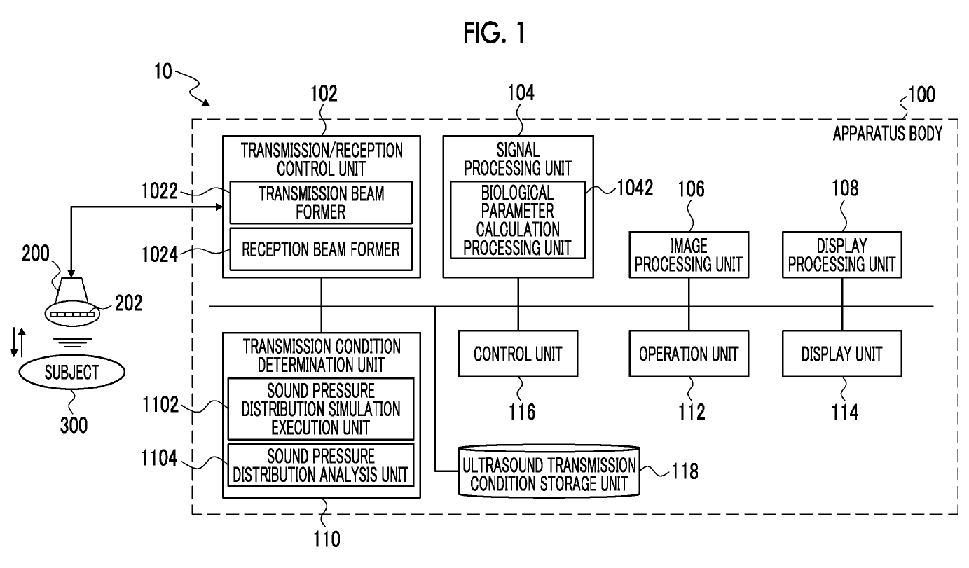

FIG. 1 is a block diagram showing a schematic configuration of an ultrasound diagnostic apparatus 10 according to the present embodiment. The ultrasound diagnostic apparatus 10 according to the present embodiment includes an apparatus body 100 and a probe 200. The apparatus body 100 is also referred to as a "console" and includes an operation unit 112 and a display unit 114, which will be described below, as a user interface. The probe 200 is a device that transmits and receives an ultrasound beam for ultrasound diagnosis. A vibration element array 202 configured by arranging a plurality of vibration elements is built in the probe 200. Each vibration element performs mutual conversion between an electric signal and an ultrasound signal by a piezoelectric effect. The probe 200 has several types such as a linear type, a sector type, and a convex type.

The ultrasound diagnostic apparatus 10 can also be referred to as an ultrasound diagnostic system. The ultrasound diagnostic apparatus 10 has a function of executing ultrasound diagnosis using the probe 200. The ultrasound diagnostic apparatus 10 according to the present embodiment particularly has a contrast-enhanced ultrasonography function. The contrast-enhanced ultrasonography is an examination in which a bubble-like contrast agent injected intravenously into a living body is caused to vibrate vigorously by an ultrasound beam, and a blood vessel is displayed with high contrast to a living tissue by receiving a nonlinear signal thereof.

In the following description, "imaging in a contrast mode" is imaging using a contrast agent, and is synonymous with imaging of a contrast agent in the contrast-enhanced ultrasonography in the present embodiment. In addition, "contrast" in an image is a difference between a bright portion and a dark portion of the image, and "high contrast" means that the difference is large. The "high contrast" in the present embodiment means that a difference in brightness between a portion into which a contrast agent is injected and a portion into which the contrast agent is not injected is large, and an ultrasound image representing a contrast agent distribution can be clearly displayed. In other words, the contrast can also be described as a ratio of an intensity of a harmonic component from the contrast agent to an intensity of a harmonic component from the living tissue.

A transmission/reception control unit 102 controls the transmission and reception of the ultrasound waves by each vibration element in the probe 200. The control includes, for example, supply of an electric transmission signal to each vibration element, amplification of an electric reception signal from each vibration element, and the like. A transmission beam former 1022 forms a transmission beam of the ultrasound waves by controlling a supply timing of a transmission signal to each vibration element. A reception beam former 1024 performs phase addition processing on a reception signal from each vibration element in the probe 200. The phase addition processing forms a reception beam. The reception beam former 1024 outputs echo data obtained along the reception beam as a result of the phase addition processing.

The signal processing unit 104 performs various types of signal processing, such as gain correction processing, logarithmic amplification processing, envelope detection processing, and filter processing, on the echo data output by the reception beam former 1024. As a result, beam data corresponding to each echo data is formed.

The signal processing unit 104 according to the present embodiment includes a biological parameter calculation processing unit 1042. The biological parameter calculation processing unit 1042 calculates a bioacoustic parameter (hereinafter, simply referred to as a "biological parameter") used for the calculation of the sound pressure distribution simulation performed by the transmission condition determination unit 110. More specifically, the biological parameter calculation processing unit 1042 acquires a set of a plurality of biological parameters (hereinafter, referred to as a "biological parameter set") representing the ultrasound propagation state in the subject by pilot transmission corresponding to the preliminary transmission and reception.

In the present embodiment, as will be described in detail below, a transmission condition estimated to be optimal is obtained in order to obtain the ultrasound image representing the favorable contrast agent distribution. In this case, it is important how accurately the sound pressure distribution is obtained. The sound pressure distribution is affected by a parameter set in a transmission condition such as a transmission frequency and an input voltage to the probe, but is also significantly changed by the biological parameter depending on a subject 300. In particular, since an acoustic attenuation rate or a sound speed included in the biological parameter greatly changes a shape of the sound pressure distribution, it is desirable that the biological parameter can be input to the sound pressure distribution simulation. Therefore, in the present embodiment, the biological parameter calculation processing unit 1042 is provided to acquire the biological parameter set and to input the biological parameter set to the sound pressure distribution simulation.

As a method of estimating the acoustic attenuation rate, for example, the technology described in JP6457107B can be used, and as a method of estimating the sound speed, for example, the technology described in JP7493481B can be used.

The image processing unit 106 has a coordinate transformation function and an interpolation function, and forms a display frame, that is, an ultrasound image based on the plurality of pieces of beam data output from the signal processing unit 104. The beam data from the signal processing unit 104 is data in a coordinate system of the beam scanning, and is composed of a plurality of data points along a direction of a beam corresponding to the beam data. The image processing unit 106 converts, for example, a signal value of each data point of the beam data into a display coordinate system, that is, a coordinate system (a rectangular coordinate system represented by a combination of an x coordinate and a y coordinate in general) of the ultrasound image. In addition, the image processing unit 106 interpolates a value of a pixel having no value from values of surrounding pixels. The image processing unit 106 forms an ultrasound image such as a B-mode tomographic image by such coordinate transformation and interpolation. In a case of the contrast-enhanced ultrasonography, the image processing unit 106 generates the ultrasound image representing the contrast agent distribution by using contrast harmonic imaging (CHI), which is a method of selectively displaying a harmonic signal generated by a nonlinear effect occurring in the ultrasound waves incident on a contrast ultrasound agent (generally, microbubbles). In the following description, the image formed by using the CHI is referred to as a "contrast image". As described above, the transmission/reception control unit 102, the signal processing unit 104, and the image processing unit 106 perform a collaborative operation to generate the contrast image in the subject 300 based on the reception information obtained from the subject 300.

The display processing unit 108 forms display screen data by combining an image or a character indicating various types of information with the ultrasound image formed by the image processing unit 106. Examples of the information combined with the ultrasound image include a region of interest (ROI) representing a display range of various display modes such as a color Doppler mode, a sample volume of a pulse Doppler mode, and a line indicating a beam on which the sample volume is positioned. The display screen data formed by the display processing unit 108 is displayed on the display unit 114.

The transmission condition determination unit 110 determines the transmission condition for transmission beam forming. The transmission condition determination unit 110 according to the present embodiment particularly determines the transmission condition suitable for imaging in the contrast mode. As will be described in detail below, the transmission condition determination unit 110 according to the present embodiment uses a plurality of transmission parameter sets set in advance in an ultrasound transmission condition storage unit 118 as the transmission condition. The transmission condition determination unit 110 includes a sound pressure distribution simulation execution unit 1102 and a sound pressure distribution analysis unit 1104.

The sound pressure distribution simulation execution unit 1102 executes the sound pressure distribution simulation by using the transmission parameter set as the transmission condition and the biological parameter set calculated by the biological parameter calculation processing unit 1042 (hereinafter, the transmission parameter set and the biological parameter set are collectively referred to as "input parameters") as the input, to estimate the sound pressure distribution. The sound pressure distribution simulation outputs the sound pressure distribution by assuming a state in which the contrast agent is injected into the subject 300. In particular, in the present embodiment, by using the biological parameter of the subject 300 to be examined as the input of the sound pressure distribution simulation, the sound pressure distribution along the depth direction in the subject can be estimated. The sound pressure distribution simulation execution unit 1102 obtains a plurality of sound pressure distributions by executing the sound pressure distribution simulation a plurality of times using different transmission parameter sets as the transmission condition.

The sound pressure distribution analysis unit 1104 selects the transmission condition estimated to be optimal for the contrast-enhanced ultrasonography to be performed on the subject 300 with reference to the plurality of sound pressure distributions obtained by the sound pressure distribution simulation execution unit 1102. Basically, the transmission condition in which a range (referred to as an "appropriate range" in the present embodiment) in which the contrast is estimated to be favorably obtained in the depth direction of the subject 300 is large is selected. In the contrast-enhanced ultrasonography, the transmission beam forming is executed by adopting the transmission condition determined by the transmission condition determination unit 110. In the present embodiment, the "depth direction" indicates a direction from a body surface of the subject 300 toward an inside of the body unless otherwise specified.

The operation unit 112 is a device operated by an operator such as a surgeon (hereinafter, also referred to as a "user") to input parameters and the like in the ultrasound diagnosis and to control the display. The display unit 114 is a device that displays an image, and is configured of, for example, a liquid crystal panel or an organic EL panel. The display screen data formed by the display processing unit 108 is displayed on the display unit 114. The control unit 116 controls the operation of each component included in the apparatus body 100 to control the execution of the ultrasound diagnostic processing.

A plurality of transmission parameter sets used in a case of executing the transmission beam forming by the sound pressure distribution simulation or the like are set in advance in the ultrasound transmission condition storage unit 118 as the measurement conditions. The transmission parameter set is created by combining a plurality of transmission parameters. The transmission parameter itself included in the transmission parameter set may have the same configuration as a transmission parameter designated in general transmission beam forming. Specific examples of the transmission parameter include an input voltage to the probe 200, a frequency, a wavefront length, a shape of the ultrasound wave (a sine wave, a sum of a plurality of sine waves, or the like), apodization, an aperture, and a focus. By changing a set value of at least one transmission parameter among these transmission parameters, a plurality of transmission parameter sets in which the set value does not completely match other transmission parameter sets are set. The plurality of transmission parameter sets having different set contents are transmission conditions that cause different transmission beams to be generated. The transmission parameter set is used as the transmission condition in a case of performing the transmission beam forming, and is also referred to as a "transmission condition" for convenience.

The functions provided by the components 102 to 116 in the apparatus body 100 are realized by a computer mounted on the apparatus body 100 and a program operating on a processor mounted on the computer in cooperation with each other. The computer may be configured to be equipped with a plurality of processors and to appropriately delegate and execute the functions provided by the components 102 to 116 to the processors. The plurality of processors may include a processor specialized in a specific processing function. The control unit 116 may be realized by a single processor or a plurality of processors. The ultrasound transmission condition storage unit 118 is realized by a storage device such as a hard disk drive (HDD) mounted on the apparatus body 100. Alternatively, the ultrasound transmission condition storage unit 118 may be realized by using an external storage device via a network.

The configuration of the ultrasound diagnostic apparatus 10 described with reference to FIG. 1 may basically have the same hardware configuration as in the related art.

### Method of Determining Transmission Condition

As described above, in the contrast-enhanced ultrasonography, the contrast agent is injected intravenously into a living body (that is, the "subject 300"), the contrast agent is caused to vibrate vigorously by the ultrasound beam, and the nonlinear signal is received as the reception information to display a blood vessel of the subject 300. However, even in a case in which the contrast-enhanced ultrasonography is performed under the same transmission condition, the vibration state of the bubble-like contrast agent varies depending on a difference in body shape of the subject 300 or the like. In addition, even in the same subject 300, the vibration state of the contrast agent may dynamically change depending on a location of a tissue in which the contrast agent is present and a condition of the subject 300 such as blood pressure and body temperature during the examination. Therefore, it is preferable to set the transmission condition suitable for the difference in the subject 300 or the physical condition of the subject 300 and perform the transmission beam forming, and as a result, to obtain a favorable contrast image in the contrast-enhanced ultrasonography.

Therefore, in the present embodiment, immediately before the contrast-enhanced ultrasonography is performed, the biological parameter is obtained as the information indicating the state of the subject 300 to be examined by performing the preliminary transmission and reception of the ultrasound waves, and the sound pressure distribution simulation is executed by using the biological parameter as the input. In the present embodiment, the transmission condition under which the optimal contrast image is obtained for the subject 300 in the contrast-enhanced ultrasonography is obtained by the sound pressure distribution simulation using the biological parameter set and various different transmission parameter sets as the transmission conditions. The optimal contrast image referred to here is a favorable contrast image with high contrast along the depth direction of the subject 300 in an imaging field of view. In addition, the favorable contrast image with high contrast basically corresponds to a case in which the sound pressure distribution obtained by the ultrasound transmission and reception is within a range of a contrast threshold value and a breakage threshold value in the imaging field of view, as will be described below. In the present embodiment, it is assumed that the favorable contrast image with high contrast is obtained in a case in which the appropriate intensity condition is satisfied, where the appropriate intensity condition is that the level of the sound pressure is within the range of the contrast threshold value and the breakage threshold value. The "contrast threshold value" is an intensity lower limit threshold value for ensuring the minimum contrast of the ultrasound image. The "breakage threshold value" is an intensity upper limit threshold value for avoiding contrast agent breakage. The contrast threshold value and the breakage threshold value can be obtained by a known method.

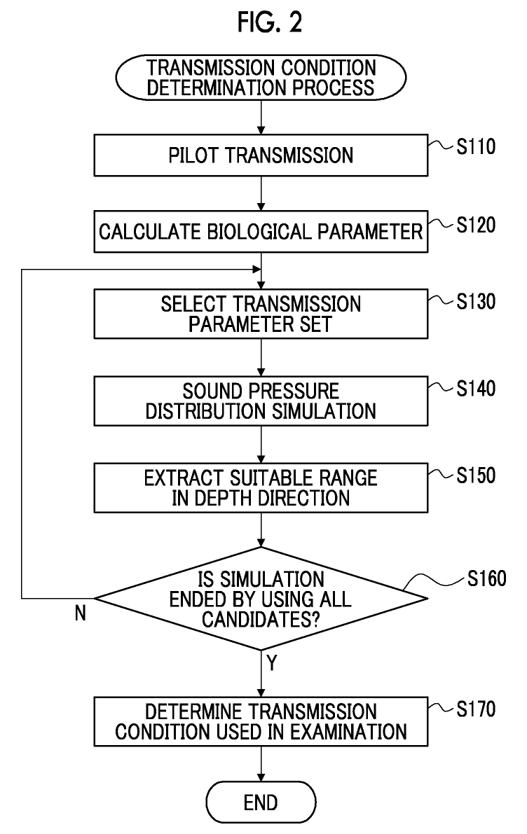

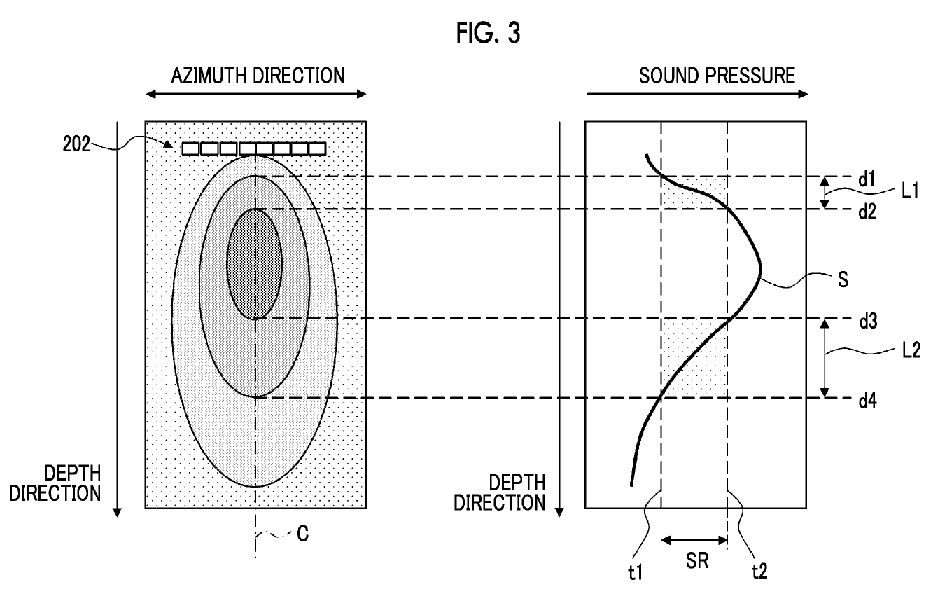

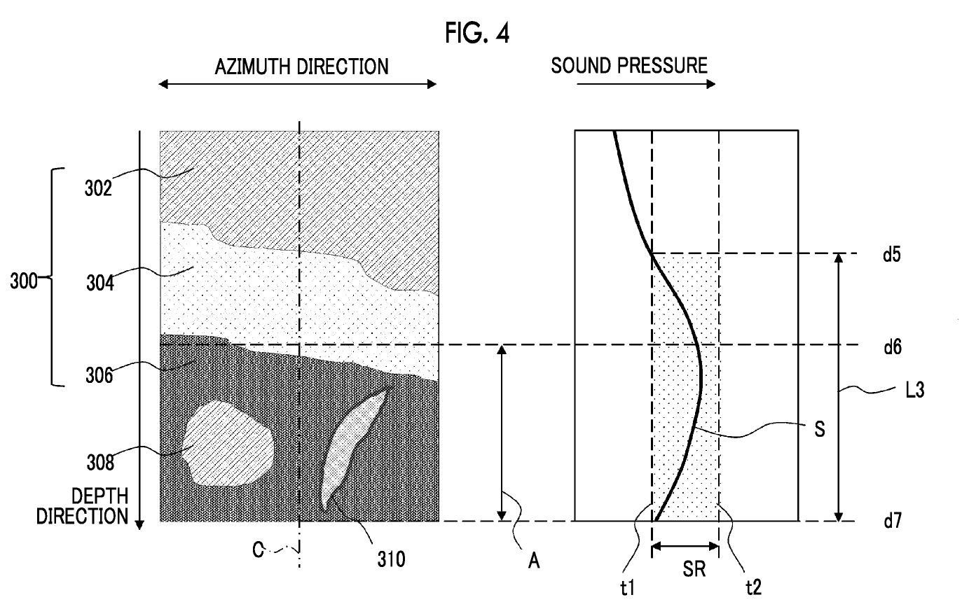

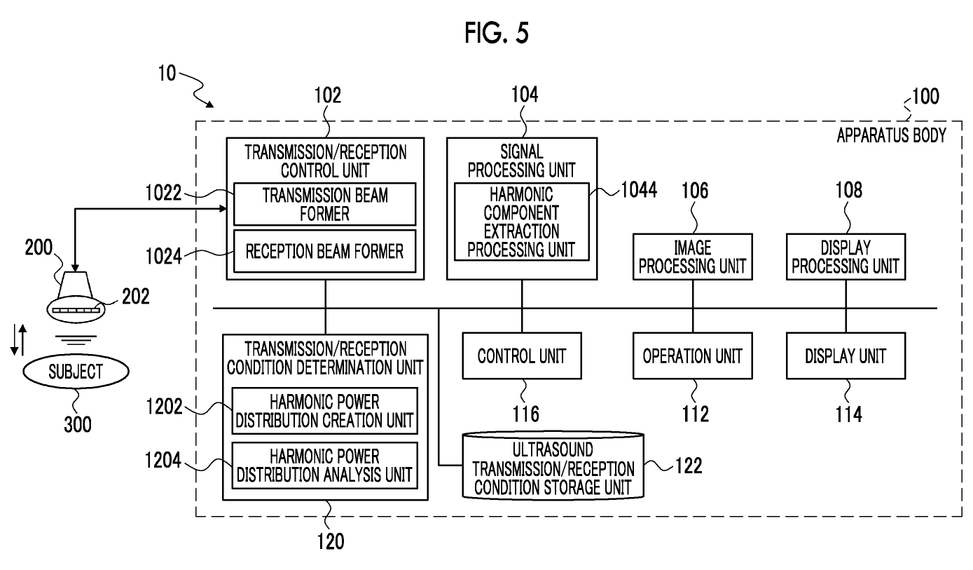

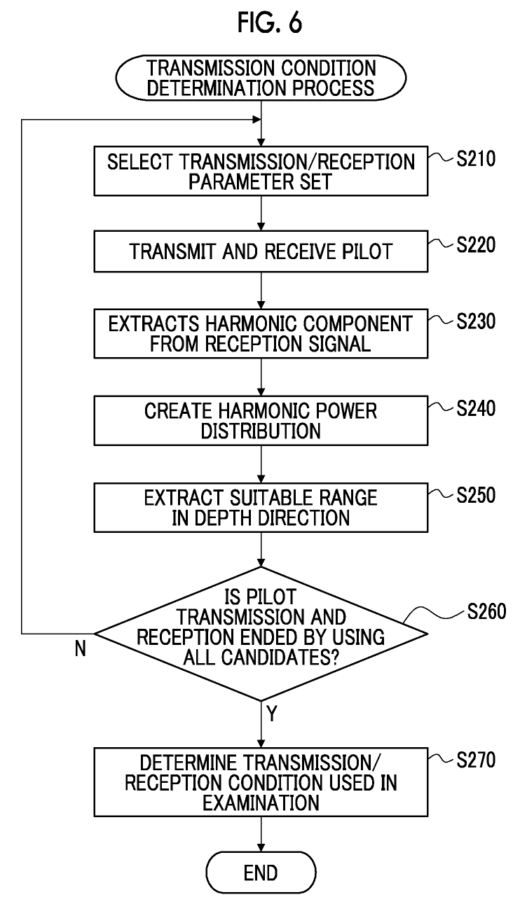

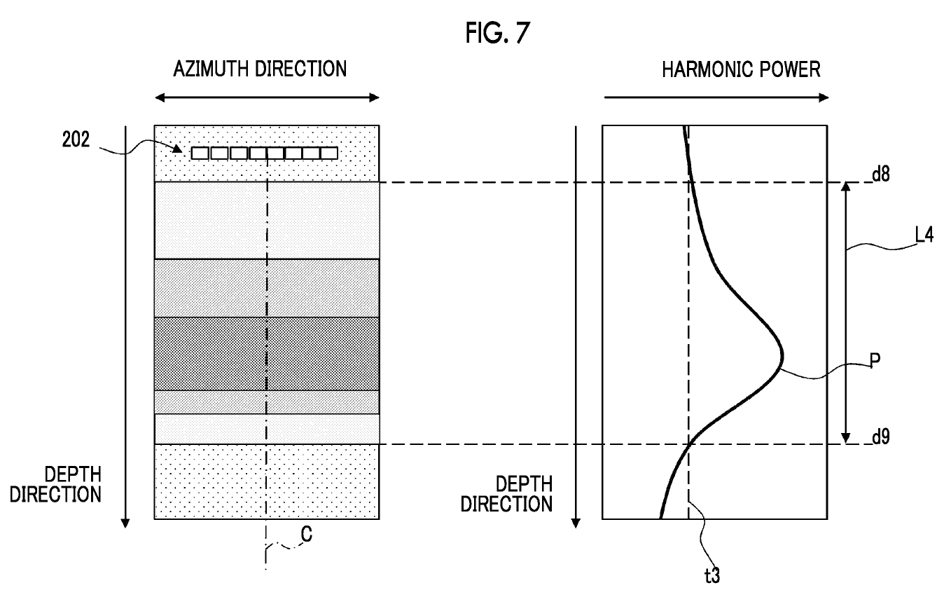

Hereinafter, the method of determining the transmission condition according to the present embodiment will be described with reference to the flowchart shown in FIG. 2.

First, the ultrasound diagnostic apparatus 10 acquires the biological parameter representing the state of the body such as the body shape of the subject 300 that is the living body or the current condition. Specifically, the ultrasound diagnostic apparatus 10 performs the pilot transmission at a first ultrasound frequency toward a predetermined site of the subject 300, for example, a site such as a tumor of an organ (step S110). The first ultrasound frequency may be a frequency used for normal ultrasound diagnosis. For example, the first ultrasound frequency may be 2 to 20 MHz.

The biological parameter calculation processing unit 1042 calculates the biological parameter from the reception signal obtained by the pilot transmission (step S120). The biological parameter is, for example, an acoustic attenuation rate, a sound speed, a thickness of subcutaneous fat, or the like at the predetermined site of the subject estimated from the reception signal. In the present embodiment, the biological parameter set is obtained by combining these biological parameters.

In a case in which the biological parameter set is obtained, the sound pressure distribution simulation execution unit 1102 selects one transmission parameter set used for the sound pressure distribution simulation from the transmission parameter sets registered in the ultrasound transmission condition storage unit 118 (step S130).

As described above, a plurality of transmission parameter sets in which at least one transmission parameter has different parameter values are registered in the ultrasound transmission condition storage unit 118. The sound pressure distribution simulation execution unit 1102 reads out the transmission parameter set that has not yet been used for the execution of the simulation from the ultrasound transmission condition storage unit 118. The reading out of the transmission parameter set is equivalent to selecting the provisional measurement condition for executing the sound pressure distribution simulation from the measurement conditions registered in the ultrasound transmission condition storage unit 118.

Then, the sound pressure distribution simulation execution unit 1102 executes the sound pressure distribution simulation by using the read transmission parameter set as the transmission condition and using the transmission condition and the biological parameter set calculated by the biological parameter calculation processing unit 1042 as the input parameters to estimate the sound pressure distribution (step S140). In the present embodiment, the sound pressure distribution indicating the intensity of the sound pressure is generated as the intensity distribution.

In this way, in a case in which one sound pressure distribution is obtained by executing the sound pressure distribution simulation once using one transmission parameter set as the transmission condition, the sound pressure distribution analysis unit 1104 extracts and obtains a range in which the favorable contrast image with high contrast is obtained in the depth direction of the subject 300 from the entire range in the depth direction by analyzing the sound pressure distribution (step S150). The range obtained here will be referred to as a "suitable range" as will be described below. Details of the processing in the sound pressure distribution analysis unit 1104 will be described below.

Subsequently, in a case in which the sound pressure distribution simulation is performed using all the transmission parameter sets registered in the ultrasound transmission condition storage unit 118 as the candidates for the transmission condition (Y in step S160), the sound pressure distribution simulation execution unit 1102 proceeds to step S170. In a case in which the transmission parameter set that has not yet been used for the execution of the sound pressure distribution simulation is present (N in step S160), the sound pressure distribution simulation execution unit 1102 proceeds to step S130 and repeats the execution of steps S140 to S150 described above.

Here, the processing in the sound pressure distribution analysis unit 1104 will be described.

The left diagram in FIG. 3 is a diagram schematically showing a sound pressure distribution acquired by executing the sound pressure distribution simulation. The right diagram in FIG. 3 is a diagram showing a level of the sound pressure obtained from the sound pressure distribution shown in the left diagram.

The left diagram in FIG. 3, the sound pressure distribution with respect to the depth direction is shown in two dimensions by transmitting and receiving the ultrasound beam from the vibration element array 202. In the left diagram in FIG. 3, a region with a darker color has a higher sound pressure. That is, in a case in which the ultrasound beam is transmitted from the vibration element array 202, the sound pressure increases toward the focal position, that is, as the depth increases. Then, it can be seen that the sound pressure decreases after passing the focal position.

The right diagram in FIG. 3 shows the level of the sound pressure extracted in one dimension along a center axis indicated by an one-dot chain line C from the sound pressure distribution shown in two dimensions in the left diagram. The sound pressure S in the right diagram FIG. 3 is a level of the sound pressure, and indicates that the level is higher in the right direction of the drawing.

In the right diagram in FIG. 3, a contrast threshold value t1 and a breakage threshold value t2 are shown, and in a case in which the sound pressure S is within a range SR between the contrast threshold value t1 and the breakage threshold value t2, the favorable contrast image with high contrast is obtained. The favorable contrast image with high contrast is a contrast image that is useful and suitable for the contrast-enhanced ultrasonography, and is referred to as a "suitable image" for convenience in the following description.

For example, in the right diagram in FIG. 3, the sound pressure S in a case in which the depth from the vibration element array 202 has not reached a depth d1 has not reached the contrast threshold value t1, and thus sufficient contrast is not obtained, that is, the suitable image is not obtained.

The sound pressure S at the depth between the depth d1 and the depth d2 from the vibration element array 202 is equal to or higher than the contrast threshold value t1 and has not reached the breakage threshold value t2, that is, is a portion within the range between the contrast threshold value t1 and the breakage threshold value t2, and thus it is shown that the suitable image is obtained in this range L1.

The sound pressure S at the depth between the depth d2 and the depth d3 from the vibration element array 202 is equal to or higher than the breakage threshold value t2, and thus it is shown that the contrast agent is broken and the suitable image is not obtained.

The sound pressure S at the depth between the depth d3 and the depth d4 from the vibration element array 202 is equal to or higher than the contrast threshold value t1 and has not reached the breakage threshold value t2, that is, is a portion within the range between the contrast threshold value t1 and the breakage threshold value t2, as in the range L1, and thus it is shown that the suitable image is obtained in this range L2.

Then, the sound pressure S in a case in which the depth from the vibration element array 202 exceeds the depth d4 has not reached the contrast threshold value t1, and thus it is shown that sufficient contrast is not obtained, that is, the suitable image is not obtained.

As described above, in the present embodiment, the ranges L1 and L2 are extracted as a range (hereinafter, referred to as a "suitable range") in which the suitable image can be obtained in the contrast-enhanced ultrasonography. The suitable range can also be referred to as a length in the depth direction, but a longer length in the depth direction results in a better suitable image.

The sound pressure distribution analysis unit 1104 calculates the suitable range as the evaluation value representing the size of the portion satisfying the appropriate intensity condition for contrast agent imaging, that is, for generating the contrast image in the sound pressure distribution by analyzing the sound pressure distribution obtained by the sound pressure distribution simulation once. That is, the sound pressure distribution analysis unit 1104 sets the level of the sound pressure S to be within the contrast threshold value t1 and the breakage threshold value t2 as the appropriate intensity condition, and calculates the portion satisfying the appropriate intensity condition, that is, the suitable range. In the present embodiment, the evaluation value is obtained as many times as the number of times of repeating steps S130 to S150, in other words, as many times as the number of transmission parameter sets registered in the ultrasound transmission condition storage unit 118.

Then, the transmission condition determination unit 110 determines the transmission condition that is the best among the obtained evaluation values, that is, the transmission condition (that is, the transmission parameter set) for obtaining the sound pressure S having the maximum total amount of the length of the suitable range in the depth direction as the transmission condition used in the contrast-enhanced ultrasonography (step S170). The determination of the transmission condition used in the contrast-enhanced ultrasonography based on the suitable range is equivalent to selecting the main measurement condition from the plurality of transmission parameter sets (that is, the measurement conditions) registered in the ultrasound transmission condition storage unit 118.

As described above, in a case in which the transmission condition is determined, the ultrasound diagnostic apparatus 10 performs the contrast-enhanced ultrasonography using the transmission condition. In the contrast-enhanced ultrasonography, the suitable image, that is, the favorable contrast image with high contrast can be displayed by using the transmission condition.

### Modification Example

The left diagram in FIG. 4 is a diagram showing a specific example of a site of the subject 300 to be examined. In the left diagram in FIG. 4, a subcutaneous fat layer 302, a muscle 304, and an organ 306 such as a liver are schematically shown. Then, it is shown that a tumor 308 and a great blood vessel 310 are present in the organ 306. The site of the subject 300 shown in the left diagram in FIG. 4 is a target of the transmission and reception of the ultrasound beam shown in the left diagram in FIG. 3, and is a target site of the sound pressure distribution simulation.

In the left diagram in FIG. 4, the sound pressure distribution as shown in the left diagram in FIG. 3 is not shown, but the right diagram in FIG. 4 shows the level of the sound pressure extracted in one dimension along the center axis indicated by the one-dot chain line C shown in the left diagram in FIG. 4. The suitable range in the right diagram in FIG. 4 can be specified by the method with reference to the right diagram in FIG. 3. That is, in a case in which the sound pressure S is within the range SR between the contrast threshold value t1 and the breakage threshold value t2, the range L3 is the suitable range in the right diagram in FIG. 4.

In the above description, the suitable range is obtained by calculation based on the entire display range of the contrast image, and when the suitable range is broader, a more favorable contrast image can be obtained. However, in the contrast-enhanced ultrasonography, the user may desire that the image of a specific range of the subject 300 is more clearly displayed even in a case in which the suitable range is slightly shortened. The transmission condition may be selected to respond to such a request of the user.

For example, in a case in which a preset (for example, an organ name such as a liver or a pancreas, or a global site name such as an abdomen or a lower limb) is input and designated, a range that the user wants to observe to some extent, in other words, a range of interest (hereinafter, referred to as a "range of interest") is determined. For example, in a case of a liver of a Japanese male, the liver surface is 3 to 4 cm and the diaphragm is 12 to 15 cm from the epidermis on average, and a range of 3 to 15 cm in which the liver is present is a desired imaging range to be drawn. As described above, the preset is input and designated by the user as one method of designating the range of interest in the depth direction in the subject.

Alternatively, the range of interest may be designated from the entire display range of the contrast image. For example, in a case in which the user wants to perform contrast only in the range in which the tumor 308 is present in the left diagram in FIG. 4, the user may manually designate the range of interest A as a range from the depth d6 to the depth d7.

In a case in which the range of interest is designated, the sound pressure distribution analysis unit 1104 obtains a proportion of a range (length) in which the suitable range includes the range of interest. Here, a proportion of the range of interest in which the range of interest overlaps the suitable range is referred to as an "overlap rate". For example, in the example shown in FIG. 4, since the range of interest A is 100% overlapped with the suitable range L3, the overlapping range (length) is A, and thus the overlap rate in the range of interest A can be calculated as an overlapping length (= A)/the range of interest (= A) = 100%.

In a case in which a plurality of transmission parameter sets having the maximum overlap rate are present, any one of the transmission parameter sets may be selected, one transmission parameter set may be selected by some selection criterion, or the user may select one transmission parameter set. As the selection criterion, for example, the transmission parameter set having the maximum suitable range may be selected. Alternatively, for example, a transmission parameter set in which an area surrounded by the depths d6 and d7 corresponding to both ends of the range of interest A, the contrast threshold value t1, and the sound pressure S is maximum on the drawing surface shown in the right diagram in FIG. 4 may be selected. Alternatively, a transmission parameter set in which an area surrounded by the depths d6 and d7, the breakage threshold value t2, and the sound pressure S is minimum may be selected. Alternatively, a threshold value t3 may be separately set between the contrast threshold value t1 and the breakage threshold value t2, and a transmission parameter set in which a range (length) exceeding the threshold value t3 is maximum may be selected.

In the present embodiment, the transmission condition used for the contrast-enhanced ultrasonography is determined as described above. The contrast-enhanced ultrasonography itself may be performed by the same method as in the related art, and thus the description thereof will be omitted. However, by performing the contrast-enhanced ultrasonography using the transmission condition determined by the above-described method, the favorable contrast image can be displayed.

In the present embodiment, in a case of executing the sound pressure distribution simulation, all the transmission parameter sets registered in the ultrasound transmission condition storage unit 118 are used, but the user may select the transmission parameter set.

In addition, a range of values that can be taken is basically determined for each transmission parameter. Therefore, the user may select the value of the transmission parameter to be used for the sound pressure distribution simulation from the range that can be taken. That is, this is equivalent to providing a function of causing the user to manually create the transmission parameter set itself. The transmission parameter set created by the user may be registered in the ultrasound transmission condition storage unit 118 or may not be registered. Alternatively, the ultrasound transmission condition storage unit 118 may be separately managed as the customized data.

In the present embodiment, the favorable contrast image can be enlarged and evaluated in the depth direction and can be displayed. In addition, the state (that is, the "biological parameter") of the subject 300 at the time of performing the contrast-enhanced ultrasonography is acquired by performing the pilot transmission before the contrast-enhanced ultrasonography is performed. As a result, the transmission condition suitable for the subject 300 at the time of performing the contrast-enhanced ultrasonography can be determined, and the favorable contrast image suitable for the subject 300 can be displayed in the contrast-enhanced ultrasonography by using the transmission condition.

In the present embodiment, the description has been made focusing on the sound pressure distribution on the center axis, that is, the sound pressure distribution in the one-dimensional direction, but the suitable range may be acquired by comprehensively considering the sound pressure distribution in the two-dimensional direction or the three-dimensional direction.

### EMBODIMENT 2

### Configuration of Ultrasound Diagnostic Apparatus

FIG. 5 is a block diagram showing a schematic configuration of the ultrasound diagnostic apparatus 10 according to the present embodiment. The configuration of the ultrasound diagnostic apparatus 10 according to the present embodiment may be basically the same as that of Embodiment 1. The ultrasound diagnostic apparatus 10 according to the present embodiment includes an internal configuration of the signal processing unit 104 and a transmission/reception condition determination unit 120 instead of the transmission condition determination unit 110. In addition, in Embodiment 1, only the transmission parameter set is registered in the ultrasound transmission condition storage unit 118, but in the ultrasound transmission/reception condition storage unit 122 according to the present embodiment, in addition to the transmission parameter set, a reception parameter set is registered.

The signal processing unit 104 according to the present embodiment includes a harmonic component extraction processing unit 1044. The harmonic component extraction processing unit 1044 extracts the harmonic component from the sound pressure distribution estimated from the reception signal obtained by the pilot transmission and reception corresponding to the preliminary transmission and reception.

The transmission/reception condition determination unit 120 according to the present embodiment determines the reception condition for the reception beam forming together with the transmission condition for the transmission beam forming. The transmission/reception condition determination unit 120 includes a harmonic power distribution creation unit 1202 and a harmonic power distribution analysis unit 1204. The harmonic power distribution creation unit 1202 creates the harmonic power distribution based on the harmonic component extracted by the harmonic component extraction processing unit 1044. The harmonic power distribution creation unit 1202 creates one harmonic power distribution for one pilot transmission and reception. The harmonic power distribution analysis unit 1204 selects the transmission/reception condition for obtaining the optimal contrast image by analyzing each of the plurality of harmonic power distributions created by the harmonic power distribution creation unit 1202.

The transmission parameter set registered in the ultrasound transmission/reception condition storage unit 122 may be the same as the ultrasound transmission condition storage unit 118 in Embodiment 1. The reception parameter itself included in the reception parameter set may have the same configuration as a reception parameter designated in general reception beam forming. Specific examples of the reception parameter include apodization, a bandpass filter threshold value (a cutoff frequency in a case of extracting the harmonic wave), a number of reception channels, and an aperture. Only one reception parameter set may be prepared. Alternatively, as in the transmission parameter set, a plurality of reception parameter sets in which the set value does not completely match other reception parameter sets may be prepared by changing the set value of at least one reception parameter among the reception parameters. The harmonic power distribution creation unit 1202 selects any one transmission parameter set registered in the ultrasound transmission condition storage unit 118 as the transmission condition and selects any one reception parameter set registered in the ultrasound transmission condition storage unit 118 as the reception condition to create the harmonic power distribution.

### Method of Determining Transmission/Reception Condition

As described above, the vibration state of the contrast agent bubbles injected intravenously into the subject 300 may dynamically change depending on a difference in body shape of the subject 300 or the like, and also depending on a location of a tissue in which the contrast agent bubbles are present or a condition of the subject 300 even in the same subject 300. Therefore, it is desired to perform the contrast-enhanced ultrasonography that is suitable for the difference in the subject 300 or the physical condition of the subject 300. Therefore, in Embodiment 1, the optimal transmission condition is determined by using the sound pressure distribution simulation. In the present embodiment, the combination of the transmission condition and the reception condition used in the contrast-enhanced ultrasonography is determined by actually executing the pilot transmission and reception, which is the preliminary transmission and reception, on the subject 300 (hereinafter, collectively referred to as a "transmission/reception condition").

Hereinafter, the method of determining the transmission/reception condition according to the present embodiment will be described with reference to the flowchart shown in FIG. 6.

First, for example, the harmonic power distribution creation unit 1202 in the transmission/reception condition determination unit 120 selects one transmission parameter set that is not used for the pilot transmission and reception described below from the transmission parameter sets registered in the ultrasound transmission/reception condition storage unit 122. In addition, any one reception parameter set is selected from the reception parameter sets registered in the ultrasound transmission/reception condition storage unit 122 (step S210).

Subsequently, the harmonic power distribution creation unit 1202 performs the pilot transmission and reception by using the selected transmission parameter set as the transmission condition and using the selected reception parameter set as the reception condition (step S220). As described above, in the present embodiment, in a case in which the combination of the transmission parameter set and the reception parameter set registered in the ultrasound transmission/reception condition storage unit 122 is used as the measurement condition, the pilot transmission and reception is executed by using the combination of the transmission parameter set and the reception parameter set selected from the ultrasound transmission/reception condition storage unit 122 as the provisional measurement condition.

Subsequently, the harmonic component extraction processing unit 1044 extracts the harmonic component derived from the contrast agent from the sound pressure distribution estimated from the reception signal obtained by the pilot transmission and reception (step S230). Then, the harmonic power distribution creation unit 1202 creates the harmonic power distribution with reference to the harmonic component extracted by the harmonic component extraction processing unit 1044 (step S240). The harmonic power distribution is a distribution related to the harmonic wave in which the sound pressure distribution is reflected, and corresponds to the intensity distribution.

In this way, in a case in which one harmonic power distribution is obtained by performing the pilot transmission and reception once using one transmission/reception parameter set as the transmission/reception condition, the harmonic power distribution analysis unit 1204 extracts and obtains a range in which the favorable contrast image with high contrast is obtained in the depth direction of the subject 300, that is, the suitable range from the entire range in the depth direction by analyzing the harmonic power distribution (step S250). Details of the processing in the harmonic power distribution analysis unit 1204 will be described below.

Subsequently, in a case in which the pilot transmission and reception is performed using all the transmission parameter sets registered in the ultrasound transmission/reception condition storage unit 122 as the candidates for the transmission condition (Y in step S260), the harmonic power distribution creation unit 1202 proceeds to step S270. In a case in which the transmission parameter set that has not yet been used for the execution of the simulation is present (N in step S260), the process proceeds to step S210, and steps S210 to S250 described above are repeatedly executed.

For example, in a case in which m transmission parameter sets are registered in the ultrasound transmission/reception condition storage unit 122 and n reception parameter sets are registered, the harmonic power distribution creation unit 1202 executes m × j (j = 1 to n) times of the pilot transmission and reception. A combination of the transmission parameter set and the reception parameter set used for the pilot transmission and reception may be selected by the user.

Here, the processing in the harmonic power distribution analysis unit 1204 will be described.

The left diagram in FIG. 7 is a diagram schematically showing a harmonic power distribution created by executing the pilot transmission and reception. The right diagram in FIG. 7 is a diagram showing a level of the harmonic power obtained from the harmonic power distribution shown in the left diagram.

In the left diagram in FIG. 7, the harmonic power distribution with respect to the depth direction is shown in two dimensions by transmitting and receiving the ultrasound beam from the vibration element array 202. In the left diagram in FIG. 7, a region with a darker color has a higher harmonic power. That is, in a case in which the ultrasound beam is transmitted from the vibration element array 202, the harmonic wave increases as the depth increases, and the bubble-like contrast agent starts to break, and thus the harmonic wave is gradually not detected.

The right diagram in FIG. 7 shows the level of the harmonic power extracted in one dimension along a center axis indicated by an one-dot chain line C from the harmonic power distribution shown in two dimensions in the left diagram in. The harmonic power P in the right diagram in FIG. 7 is a power, in other words, a level of the intensity of the harmonic wave, and indicates that the level is higher in the right direction of the drawing. The intensity can also be referred to as sensitivity.

In the right diagram in FIG. 7, a harmonic threshold value t3 is shown. The harmonic threshold value t3 is an intensity lower limit threshold value indicating a lower limit of the intensity for ensuring the desired contrast of the contrast image. In a case in which the harmonic power P is equal to or higher than the harmonic threshold value t3, the favorable contrast image with high contrast, that is, the suitable image is obtained.

It is considered that the contrast in the contrast mode and the breakage of the contrast agent can be determined from the value of the harmonic wave, and thus the harmonic threshold value t3 is set instead of setting the contrast threshold value t1 and the breakage threshold value t2 as in Embodiment 1. The harmonic threshold value t3 is a threshold value that can be estimated to be able to obtain the desired contrast in a case in which the harmonic wave equal to or higher than the harmonic threshold value t3 is obtained. In a case in which the contrast agent vibrates appropriately, more harmonic waves can be obtained. In addition, in a case in which the contrast agent is broken, the value of the harmonic component is low.

For example, in the right diagram in FIG. 7, the harmonic power P in a case in which the depth from the vibration element array 202 has not reached a depth d8 has not reached the harmonic threshold value t3, and thus it is shown that sufficient contrast is not obtained, that is, the suitable image is not obtained.

The harmonic power P at the depth between the depth d8 and the depth d9 from the vibration element array 202 is equal to or higher than the harmonic threshold value t3, and thus it is shown that the suitable image is obtained in a range L4 from the depth d8 to the depth d9.

Then, the harmonic power P in a case in which the depth from the vibration element array 202 exceeds the depth d9 has not reached the harmonic threshold value t3, and thus it is shown that sufficient contrast is not obtained, that is, the suitable image is not obtained.

As described above, in the suitable range in the contrast-enhanced ultrasonography, the suitable image is obtained in the range L4 in the example shown in the right diagram in FIG. 7, and the longer the length of the range L4 in the depth direction, that is, the larger the total amount of the suitable range, the better the resulting suitable image quality.

As described above, in the present embodiment, the appropriate intensity condition in which the harmonic power P is equal to or higher than the harmonic threshold value t3 is set. Therefore, the portion satisfying the appropriate intensity condition is a portion in which the harmonic power P is equal to or higher than the harmonic threshold value t3 as in the range L4. In the present embodiment, the suitable range is calculated as the evaluation value representing the size of the portion satisfying the appropriate intensity condition for generating the contrast image.

The harmonic power distribution analysis unit 1204 obtains a range in which the suitable image is obtained in the depth direction of the subject 300 from the harmonic power distribution as shown in the right diagram in FIG. 7. Then, the transmission/reception condition determination unit 120 determines the transmission/reception condition (that is, the combination of the transmission parameter set and the reception parameter set) for obtaining the range (that is, the total amount of the length in the depth direction) that is the maximum among the ranges in which the obtained suitable image is obtained as the transmission/reception condition used in the contrast-enhanced ultrasonography (step S270). The determination of the transmission/reception condition used in the contrast-enhanced ultrasonography based on the suitable range is equivalent to selecting the main measurement condition from the plurality of transmission/reception parameter sets (that is, the measurement conditions) registered in the ultrasound transmission/reception condition storage unit 122.

As described above, in a case in which the transmission/reception condition is determined, the contrast-enhanced ultrasonography is performed using the transmission/reception condition. In the contrast-enhanced ultrasonography, the suitable image, that is, the favorable contrast image with high contrast can be displayed by using the transmission/reception condition. In the present embodiment, the harmonic component included in the reception signal can be effectively extracted and imaged instead of directly displaying the reception signal. In other words, a blood vessel portion to be imaged into which the contrast agent is injected can be displayed bright, and other biological portions can be displayed dark.

In the present embodiment, the range of interest may be designated by the user as in the case of Embodiment 1, although the description thereof will be omitted.

What is claimed is:

1. An ultrasound diagnostic apparatus comprising:
a processor,
wherein the processor is configured to:
select a provisional measurement condition from a plurality of measurement conditions that cause different transmission beams to be generated;
generate an intensity distribution by performing a simulation or preliminary transmission and reception based on the provisional measurement condition, the intensity distribution being a sound pressure distribution along a depth direction in a specific subject or a distribution related to a harmonic wave in which the sound pressure distribution is reflected;
calculate an evaluation value representing a size of a portion satisfying an appropriate intensity condition for contrast agent imaging in the intensity distribution;
select a main measurement condition from the plurality of measurement conditions based on a plurality of evaluation values each corresponding to the plurality of measurement conditions; and
generate an ultrasound image representing a contrast agent distribution in the specific subject based on reception information obtained from the specific subject by performing transmission and reception for contrast-enhanced ultrasonography based on the main measurement condition.

2. The ultrasound diagnostic apparatus according to claim 1,
wherein the appropriate intensity condition includes an intensity lower limit threshold value for ensuring a minimum contrast of the ultrasound image and an intensity upper limit threshold value for avoiding contrast agent breakage, and
the portion satisfying the appropriate intensity condition is a portion of the sound pressure distribution that falls between the intensity lower limit threshold value and the intensity upper limit threshold value.

3. The ultrasound diagnostic apparatus according to claim 1,
wherein the measurement condition is a transmission parameter set that is a transmission condition for generating a transmission beam.

4. The ultrasound diagnostic apparatus according to claim 3,
wherein the processor is configured to:
perform preliminary transmission and reception for obtaining a biological parameter set representing an ultrasound propagation state in the specific subject based on the reception information obtained from the specific subject; and
execute the simulation by using the transmission parameter set and the biological parameter set as input parameters to estimate the sound pressure distribution along the depth direction in the specific subject.

5. The ultrasound diagnostic apparatus according to claim 1,
wherein the appropriate intensity condition has an intensity lower limit threshold value for ensuring a desired contrast of the ultrasound image, and
the portion satisfying the appropriate intensity condition is a portion of the distribution related to the harmonic wave that is equal to or higher than the intensity lower limit threshold value.

6. The ultrasound diagnostic apparatus according to claim 1,
wherein the measurement condition is a combination of a transmission parameter set, which is a transmission condition in a case of performing the preliminary transmission and reception and the transmission and reception for the contrast-enhanced ultrasonography, and a reception parameter set that is a reception condition.

7. The ultrasound diagnostic apparatus according to claim 1,
wherein the processor is configured to calculate the evaluation value based on a display range of the ultrasound image or a range of interest in the depth direction in the specific subject.
